(19) United States
(12) Patent Application Publication
(10) Pub. No.: US 2012/0095363 A1
(43) Pub. Date: Apr. 19, 2012

(54) COMPOUND TACTILE STIMULUS DEVICE SYSTEM

(76) Inventor: Joseph P. Garner, Brisbane, CA (US)

(21) Appl. No.: 13/145,811

(22) PCT Filed: Jan. 22, 2010
(86) PCT No.: PCT/US10/21805

§ 371 Date: Jan. 3, 2012

Related U.S. Application Data
(60) Provisional application No. 61/146,512, filed on Jan. 22, 2009.

(51) Int. Cl. A61B 19/00

(52) U.S. Cl. 600/557

(57) ABSTRACT

A system for presenting compound tactile stimuli to a test subject includes a compound tactile stimulus device comprised of a plurality of overlapping stimulus presentation members, each stimulus presentation member including one or more stimulus exemplars related by a specified dimensional property. The device includes manual or automated means for moving and aligning the stimulus presentation members relative to one another to form a predetermined compound tactile stimulus comprised of at least two tactile stimulus exemplars, including at least one exemplar having one or more open spaces allowing the test subject to discriminate between the stimulus exemplars underlying the compound tactile stimulus by extending one or sensory members through the one or more open spaces. The system may include the compound tactile stimulus device integrated into an automated living environment providing automated compound tactile stimulus presentation, including automatic generation of behavioral data under conditions that are less susceptible to experimental bias by minimizing artificial or unnatural testing conditions that may otherwise confound the results.

This application claims the benefit of priority under 35 U.S.C. §119(e) to U.S. Provisional Application No. 61/146,512, filed Jan. 22, 2009, which is hereby incorporated by reference in its entirety.

### FIELD OF THE INVENTION

This invention relates to the field of experimental animal testing. More specifically, this invention relates a compound tactile stimulus device system for use with animal test subjects.

### BACKGROUND

Paradigms in the behavioral sciences, both animal and human, involve the presentation of stimuli to experimental test subjects. For instance, a stimuli might be presented and the subject required to make one or more differential responses on the basis of the stimulus (operant paradigms), a stimulus might indicate which corridor to choose in a maze (maze paradigms), or a stimulus might be paired with the presentation of other stimuli, reward, or punishment (associative learning paradigms). Such paradigms are commonly used in, for example, animal behavior (ethology), experimental psychology, neurobiology, neuropsychology, behavioral phenotyping, drug development, pharmacology, genetic engineering, and psychiatric research.

Traditionally, these stimuli are visual (e.g., lights or shapes on a computer screen), auditory (e.g. tones), and sometimes olfactory. For many paradigms two stimuli are presented that differ in some way. Such differences may be quantitative (e.g., a light that flashes fast versus slow, or a high versus low pitched tone), or qualitative (e.g., a square versus triangular shape, or a pepper versus a ginger odor).

Modern behavioral experiments are often automated using computer-controlled equipment (e.g., the computer-controlled “Skinner box” experiments). In addition, many cutting-edge experiments require the presentation of qualitatively different ‘compound’ stimuli that vary in two or more contextually distinct properties (termed dimensions) (e.g., shapes overlaid with different lines). Different variations within a dimension (e.g., square versus triangle within the shape dimension) are termed exemplars.

The presentation of qualitatively different compound stimuli has been almost exclusively constrained to visual stimuli presented on a computer screen—a method that is well suited to primates and humans. However, this method is poorly suited, for example, to rodent species with poorly developed vision (for example, many widely used mouse strains) or species or strains that lose visual acuity as they age (for example, but not limited to, some widely used mouse strains). Thus, for instance, mice do not see the same color range as humans, their visual acuity is 1/50^{th} that of humans, the lens of their eye is fixed-focus and thus unable to adjust to the distance of an object; and generally their visual system is designed to detect movement at a distance, rather than to discriminate objects at close range. Although qualitative auditory stimuli can be presented (e.g., the sound of a whistle versus the sound of a horn), additional dimensions of relevance to the animal are hard for humans to identify for experimental manipulation, not least because species differ greatly in not only the basic frequencies of sound detected, but also in the properties of sound that they can detect (e.g., the bandwidth of individual filters and thus the pitches they can discriminate, and the tuning of their auditory system to detect particular forms of frequency and amplitude modulation). Thus as with vision, although humans might not hear frequencies other animals can hear, the complexity of auditory processing is highly developed compared to many other animals (including mice).

Learning, cognitive, and neuropyschological paradigms in animals are typically time consuming, as they often involve prolonged periods of training and tens to hundreds of thousands of animal testing trials. Furthermore, behavioral paradigms are generally hampered when the responses required of the animal, or the stimuli they are required to respond to are unduly artificial with respect to their natural behavior (Young et al., Anim. Behav., 47:1488-90, 1994). This effect essentially masks the true learning, cognitive, or neuropsychological processes being assessed, and becomes increasingly severe as the complexity or subtlety of the process under study increases (Garner et al., Behav. Brain Res., 173: 53-61, 2006). Generally speaking, at the level of complexity required to perform clinically relevant paradigms, attention to this issue is paramount. For instance, in mouse maze experiments to assess measures of frontal brain function of clinical relevance in humans, an experimenter may manually present compound qualitative stimuli in the form of a small cup with different outside textures filled with different inner substrate, and marked with different odors. Although mice excel at processing these stimuli (which are tuned to their sensory biology), they are unable to perform the same complex task when stimuli are presented visually (Garner et al., Behav. Brain Res., 173: 53-61, 2006). Further, such mouse maze experiments may typically require the experimenter to manually place a new stimulus object in the maze for each trial. Such methods for assessing clinically relevant measures require a series of time-consuming trials that are not readily amenable to automation.

Behavioral phenotyping approaches are typically characterized by rapid assessment of large numbers of animals for behavior mutants, drug screening for behavior effects, screening of genetically modified animals, and large-scale dose response pharmacological experiments. Behavioral phenotyping, by maximizing throughput, typically sacrifices the controls, complexity, and resolution of other behavioral methods, and are generally unable to distinguish between clinically relevant deficits, including a wide variety of learning and memory deficits specific to differential human diagnoses. Behavioral phenotyping approaches are sensitive to confounding effects both in the environment and within the animal. Confounding environmental effects include the time of day when the experiment is done, as well as negative effects due to experimenter interactions. Confounding effects within the animal may be illustrated in behavioral phenotyping measures of anxiety in animals, which may conflict fear against exploratory drive, whereby differences between animals in these measures may equally well be due to differences in anxiety, fear, and/or exploratory drive, which present altogether different issues from a human clinical standpoint. Consequently, behavioral phenotyping has poor external predictive validity in human trials; for example, only 8% of compounds showing behavioral effects in mice actually show clinical efficacy in humans (Kola & Landis, Nat. Rev. Drug. Discov., 3: 711-716, 2004).

Behavioral phenotyping methodologies explicitly disregard the issue of animal sensory biology, because more clinically relevant measures are too time consuming, and are not amenable to automation. Behavioral phenotyping methodologies are further hampered by the use of inappropriate controls, which contributes to the high level of false positive results (Richter et al., Nature Meth., 6(4):257-261, 2009). This is especially the case, for example, where interactions with the experimenter, testing during the animal's sleep cycle, and other stressors in the testing environment, which are known to markedly degrade many different kinds of behavioral data (Hossain et al., Genes Brain Behav., 3:167-177, 2004), are not corrected for, in large part, because suitable experimental designs to control for these effects are time-consuming.

In the context of high-throughput behavioral studies involving animals, such as mice, for example, there is a need for high-throughput behavioral study methodologies which combine the throughput of behavioral phenotyping with the clinical relevance of other methods. Such methodologies might obtain behavioral measurements using natural responses (e.g., searching for food) to natural stimuli (e.g., texture and odor), which are naturally utilized in the normal foraging behavior of wild mice, late in the daylight hours or night when mice are normally active, under circumstances whereby the execute tasks require little or no training by the mouse. Further, such methodologies would ideally benefit from the integration of an automated compound tactile stimulus device in a home cage setting involving minimum human intercession so as to reduce the impact of the experimentor and test environment stressors from the behavioral measurements.

Accordingly, the present invention provides devices for presenting compound tactile stimuli, along with systems for integrating such devices in automated environmental living systems. To address the above problems and needs, the present invention provides a compound tactile stimulus presentation system containing a compound tactile stimulus device. The compound tactile stimulus device may be integrated into an automated 24 hour a day living environment providing automated compound tactile stimulus presentation. Integration of the compound tactile stimulus device into this system allows animals to unwittingly participate in behavioral experiments during normally active time periods in which they are motivated to execute normal tasks and behaviors, such as foraging, whereby they work for their food and other resources, while generating behavioral data during their daily routine.

### SUMMARY

A system for presenting compound tactile stimuli to a test subject includes a compound tactile stimulus device comprised of a plurality of overlapping stimulus presentation members, each stimulus presentation member including one or more stimulus exemplars related by a specified dimensional property. The device includes means for moving the stimulus presentation members relative to one another, aligning the exemplars from one presentation member with exemplars in other presentation member(s) to form a pre-determined compound tactile stimulus comprised of at least two tactile stimulus exemplars. One or more of the tactile stimulus exemplar include one or more open spaces allowing the test subject to investigate (and discriminate between) the stimulus exemplars underlying the compound tactile stimulus by extending one or sensory members through the one or more open spaces.

Presentation of compound tactile stimuli may be limited to tactile exemplars or they may additionally include olfactory, auditory, or visual exemplars, or combinations thereof. In one embodiment, the compound tactile stimulus include front, center, and back tactile stimulus exemplars presented from front, center, and back stimulus presentation members in which each of the front and center tactile exemplars have one or more open spaces. In another embodiment, the stimulus presentation members may be mounted behind a panel containing a window focusing an animal's attention and restricting its physical access to the compound tactile stimulus through the window via open spaces in the stimulus exemplars.

The compound tactile stimulus presentation system may employ manual or automatic means for moving and aligning the stimulus presentation members to present a pre-selected compound tactile stimulus. An automated compound tactile stimulus device may include positional means for determining the position of the stimulus presentation members; driving means for driving the stimulus presentation members; controlling means for controlling the position and movement of the stimulus presentation members; signaling means for coordinating execution of the positional, driving, and controlling means, and investigation means for determining whether the compound tactile stimulus has been investigated by an animal. By way of example, an automated compound tactile stimulus device may include a positional switch for monitoring the positions of the stimulus presentation members, a controlling device operatively coupled to one or more motors for driving the stimulus presentation members, interface electronics operatively coupled to the controlling device for coordinating signals between the controlling device, one or more motors to control movement of the stimulus presentation members; and an infra-red (IR) beam for determining that a compound tactile stimulus has been investigated by an animal.

The stimulus presentation members may be rotatable or laterally extendable relative to one another. Additionally, the stimulus presentation members may be configured as overlapping members in a triangular or co-axial arrangement. The stimulus presentation members may be configured as a plurality of stimulus wheels, each wheel including one or more stimulus exemplars related by a specified dimensional property. By way of example, the compound tactile stimulus device may include a front stimulus wheel, a center stimulus wheel, and a rear stimulus wheel. Exemplars in the front stimulus wheel may include cut outs varying by shape; exemplars in the center stimulus wheel may include various patterns of lines against a background comprising one or more open spaces, exemplars in the rear stimulus wheel comprise exemplars comprising variously textured substrates.

In another aspect, the compound tactile stimulus presentation system may further include housing in which the compound tactile stimulus device is enclosed. The housing may include a home cage, skinner box, maze, or combination thereof. In addition, the system may include means for automatically delivering rewards, punishments, or neither, based on the nature of the pre-selected compound tactile stimulus and the animal's response. In one embodiment, the system includes the compound tactile stimulus device integrated into an automated living environment system. In another embodiment, the system provides an automated living environment in which pre-determined compound tactile stimuli, rewards, and/or punishments are presented under control of one or more controlling devices, and behavioral data is automatically generated under control of the controlled device(s).

In a further aspect, the present invention provides a method for utilizing the compound tactile stimulus presentation system for behavioral studies. In one embodiment, an animal behavioral test includes exposing an animal to a compound tactile stimulus presentation system according to the present invention so that at least one pre-determined compound tactile stimulus is presented to the animal, whereby the stimulus indicates one or more locations that may or may not contain an accessible reward, punishment, or neither. Conditions suitable for enabling the animal to receive the reward, punishment, or neither, depending on the animal's response to the compound stimulus are provided. These steps are repeated, altering the compound tactile stimulus as appropriate until a suitable number of tests have been obtained in accordance with the underlying behavioral test or behavioral paradigm(s) which are being investigated.

In another embodiment, an animal behavioral test is initiated by placing an animal in a compound tactile stimulus presentation system containing home cage containing a first one-way door leading to a first compartment containing the compound tactile stimulus device. The compound tactile stimulus device is positioned between a second one-way door leading into a second compartment and a third one-way door leading into a third compartment. One of the first and second compartments contains an accessible reward or punishment. The animal is allowed to enter the first compartment through the first one-way door. The animal is exposed to the compound tactile stimulus device presents a pre-determined compound tactile stimulus indicating the location of an accessible reward or punishment in either the second compartment or the third compartment. Upon investigation of the compound tactile stimulus, the animal enters either the second or third compartment through the second or third one-way door, respectively. The second and third compartments lead back to the home cage by way of a fourth or fifth one-way door, respectively. Upon entry back into a home cage, the animal can repeat the above steps, in which case the compound tactile stimulus may be retained or modified in any subsequent trial.

By integrating an automated compound tactile stimulus device into suitable housing, the present invention may provide an automated living environment system providing automated compound tactile stimulus presentation, including automatic generation of behavioral data under conditions that are less susceptible to experimental bias adversely affected by artificial or unnatural testing conditions.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The present invention will become more fully understood from the detailed description given herein below and the accompanying drawings which are given by way of illustration only, and are not limitative of the present invention and wherein:

FIG. 1 is an exemplary view of a manually operated compound tactile stimulus device.

FIG. 2 illustrates exemplary stimulus presentation plates for use in the manually operated compound tactile stimulus device depicted in FIG. 1.

FIG. 3 is an exemplary mock-up of an automated compound tactile stimulus device with front panel absent.

FIG. 4 is an exemplary view of a possible configuration of stimulus wheels with front panel absent.

FIG. 5 is an exemplary view of a configuration of stimulus wheels with a front panel.

FIG. 6 is an exploded view of a possible configuration of stimulus wheels relative to a front panel.

FIG. 7 is a perspective view of three exemplary stimulus wheels and an exemplary compound tactile stimulus.

FIG. 8 is an exemplary view of components of an automated compound tactile stimulus device.

FIG. 9 is an exemplary view illustrating motor and control means.

FIG. 10 is an exemplary view of another possible configuration of an automated compound tactile stimulus device with front panel absent.

FIG. 11 is an exemplary top view of the device of FIG. 10.

FIG. 12 is another exemplary view of a stimulus wheel.

FIG. 13 is another exemplary view of a stimulus wheel.

FIG. 14 is another exemplary view of a stimulus wheel.

FIG. 15 is another exemplary view of a stimulus wheel with an exploded view of a textured section.

FIG. 16 is an example view of the overlapping region presented to the test subject.

FIG. 17A is an exemplary view of an automated linear drive configuration of a compound tactile stimulus device according to another embodiment.

FIG. 17B is a close-up view illustrating further aspects of the embodiment in FIG. 17A.

FIG. 17C is an exemplary view of a compound tactile stimulus formed by the embodiment in FIGS. 17A and 17B.

FIG. 18 illustrates exemplary stimulus presentation plates for use in the embodiment depicted in FIG. 17.

FIG. 19 is diagram of a compound tactile device system including a maze connected to a home-cage.

FIG. 20 illustrates the results of a mouse tactile discrimination test.

### DETAILED DESCRIPTION

### Definitions

In order to provide a clear and consistent understanding of the specification and claims, the following definitions are provided.

As used herein, the term “stimulus exemplar” refers to a stimulus related by a specified dimensional property. The dimensional property may be related by touch (tactile exemplars), smell (olfactory exemplars), sound (auditory exemplars), or sight (visual exemplars). A specified dimensional property may further define generic tactile, auditory, olfactory and visual exemplars by additional dimensional characteristics related to material form, smell, pitch, and volume. Although an exemplar may be assessed by some species or individuals in two sensory modalities (for example, tactile and visual), for purposes of this application such an exemplar would be considered a single exemplar, inasmuch as the tactile and visual dimensions are not dissociable.

The term “compound stimulus” refers to a compound stimulus comprised of at least two independent, dissociable stimulus exemplars, each exemplar being defined by a specified dimensional property. A compound stimulus according to the present invention may include tactile stimulus exemplars, auditory stimulus exemplars, olfactory stimulus exemplars, visual stimulus exemplars, or combinations thereof.

The term “compound tactile stimulus” refers to a compound stimulus comprised of at least two tactile stimulus exemplars, including at least one tactile stimulus exemplar exhibiting one or more open spaces. The compound tactile stimulus may be limited to tactile stimulus exemplars, or it may additionally include one or more auditory exemplars, olfactory exemplars, visual exemplars, or combinations thereof.

In one embodiment, a system for presenting compound tactile stimuli to a test subject includes a compound tactile stimulus device comprised of a plurality of overlapping stimulus presentation members, each stimulus presentation member including one or more stimulus exemplars related by a specified dimensional property. The device includes means for moving the stimulus presentation members relative to one another, aligning the exemplars from one presentation member with exemplars in other presentation member(s) to form a pre-determined compound tactile stimulus comprised of at least two tactile stimulus exemplars. Thus, each stimulus presentation member will contribute a particular exemplar to the compound tactile stimulus. One or more of the tactile stimulus exemplars include one or more open spaces allowing the test subject to investigate (and discriminate between) the stimulus exemplars underlying the compound tactile stimulus by extending one or sensory members through the one or more open spaces. Exemplary sensory members include but are not limited to whiskers, paws, fingers, lips, tongues, genitalia or other appendages are used for tactile investigation.

Stimulus exemplars sharing a common dimensional property may be related by touch, smell, sight, or combinations thereof. Thus, a stimulus presentation member may include tactile exemplars, auditory exemplars, olfactory exemplars, visual exemplars, or combinations thereof. Tactile exemplars may be related by a specified dimensional property, such as shape, size, number, softness, hardness, etc. For example, one set of tactile exemplars may be defined by various cut out shapes containing uniform open spaces; a second set may be defined by different patterns, grids, or meshes of material elements over a background of one or more open spaces, including various patterns of lines or bars over an open background; and a third set may be defined by different textured materials against a solid background. Auditory exemplars may be defined by pitch, volume intensities, frequency modulation or amplitude modulation. Olfactory exemplars may be defined by different smells.

The compound tactile stimuli are preferably designed for use in animal testing by species, strains, or individuals, with sensory modalities particularly suited for discriminating the different stimulus exemplars in a given compound tactile stimulus. For example, experiments employing compound tactile stimuli may preferably employ animals suitably adapted for discriminating between the different tactile stimulus exemplars in a compound tactile stimulus, especially any animals where whiskers, paws, fingers, lips, tongues, genitalia or other appendages are used for tactile investigation. Exemplary animals for discriminating tactile exemplars include but are not limited to rodents, such as mice, rats, voles, gerbils, hamsters, and guinea pigs; cats; primates, including monkeys, chimpanzees, and humans; octopi; and insects, including cockroaches.

The compound tactile stimulus presentation system may employ manual or automatic means for moving and aligning the stimulus presentation members to present a pre-selected compound tactile stimulus. An automated compound tactile stimulus device may include means for sensing and encoding the position of the stimulus presentation members, means for determining the position of the stimulus presentation members, means for driving the stimulus presentation members, means for controlling the position and movement of the stimulus presentation members, signaling means for coordinating the activities of the automation means; and means for means for determining that the compound tactile stimulus has been investigated by an animal.

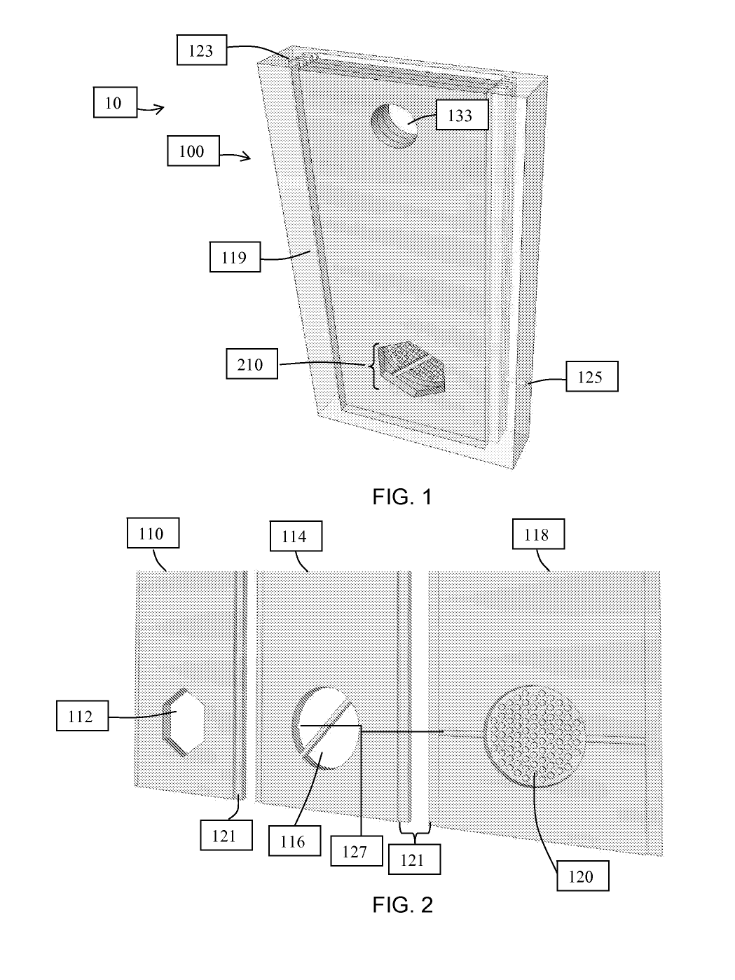

FIGS. 1 and 2 illustrate exemplary views of a compound tactile presentation system 10 containing a manually operated compound tactile stimulus device 100. The manually operated device 100 in FIGS. 1 and 2 includes a plurality of stimulus presentation members in the form of overlapping plates 110, 114, and 118, each plate defined by different tactile stimulus exemplars 112, 116, and 120. The plates 110, 114, and 118 are aligned in a frame 119 by means of flanges 121 that slide in grooves 123 in the frame 119 to form a compound tactile stimulus 210 where their exemplars 112, 116, and 120 overlap. Near the top of each plate is hole 133 to facilitate handling, removal, and insertion of the plates. The device 100 presents the compound tactile stimulus 210 so that the test subject can investigate and discriminate between the different exemplars underlying the compound tactile stimulus 210. The center 114 and back 118 plates have an inlaid groove 125 aligning with one another and the frame 119 to form a channel 127 for an infra-red (IR) beam (not shown) to determine whether the compound tactile stimulus 210 has been investigated by the animal. In this case, the IR beam serves to detect investigation of the back plate 118, confirming that the animal has investigated the front two stimuli. The channel 127 is semi-circular in cross section, extending into the center 114 and back 118 plates to form a circular cross section when the center 114 and back 118 plates align. Alternatively, the device 100 may employ a single circular channel 127 in the back 118 plate.

FIG. 2 illustrates exemplary stimulus presentation plates 110, 114, and 118 for use in the embodiment depicted in FIG. 1. The plates are aligned in the frame 119. The center 114 and back 118 plates have an inlaid groove 125 aligning with one another and the frame 119 to form a channel 127 for an infra-red (IR) beam 129 to detect investigation of the compound tactile stimulus 210 by the animal. The back plate 118 includes a textured substrate 120 against a solid background.

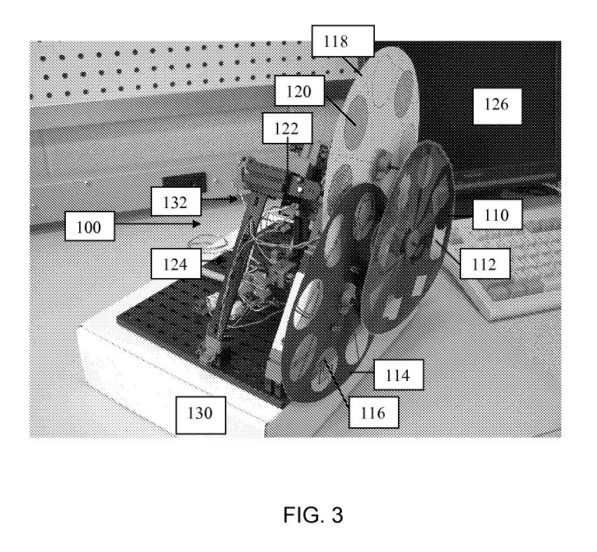

FIG. 3 is an exemplary automated device to present tactile stimuli 10. The automated device 100 in FIG. 3 includes four basic components: a plurality of stimulus presentation members 110, 114, and 118, each member including a plurality of stimulus exemplars 112, 116, and 120; a motor 122; a controlling device 126; and interface electronics 124. The automated device may include additional, fewer, or alternate components.

In one embodiment, as shown in FIG. 3, stimulus presentation members 110, 114, and 118 may be in the form of wheels, each containing a different set of tactile exemplars for forming the compound tactile stimulus 210. The device 100 presents the compound tactile stimulus 210 so that the test subject can investigate and discriminate between the different exemplars 112, 116, and 120 underlying the compound tactile stimulus 210. In one embodiment, both the first 110, and second 114 presentation member provide opening(s) permitting physical access to a third presentation member 116 therebehind. For example, a first, outer-most stimulus wheel 110 may feature a first set of exemplars as cut-outs 112 in various shapes defining openings in the wheel. A second stimulus wheel 114 behind the first wheel may feature a second set of exemplars as various mesh materials 116 in, for example, in various line patterns distinctively arrayed among open spaces. The third stimulus wheel 118 behind the second wheel may feature a third set of exemplars as different textured substrates 120 against a solid background. When layered, stimulus presentation members 110, 114, and 118 may present a compound tactile stimulus 210 where their exemplars overlap. In FIG. 3, the compound tactile stimulus 210 allows physical access through the first and second wheels so as to permit the test subject to touch all three wheels at once 210.

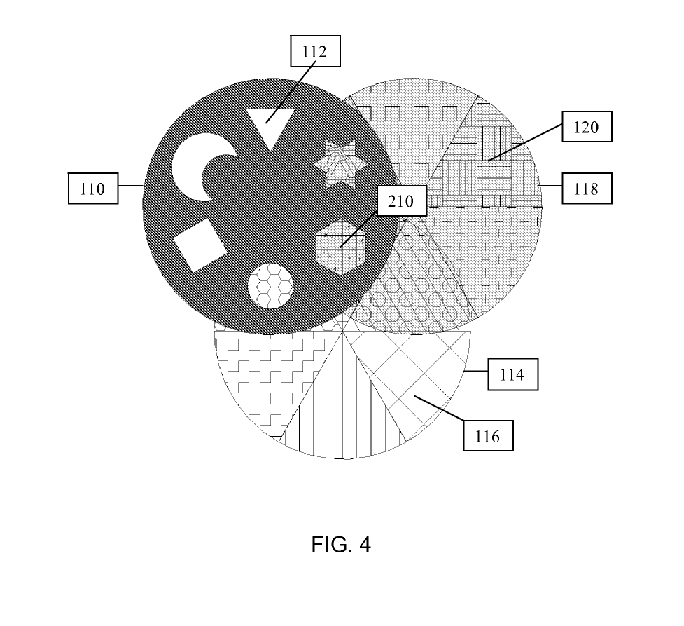

The stimulus presentation members may be arranged in a partially overlapping, triangular arrangement of the first 110, second 114, and third 118 stimulus wheels as shown in FIG. 4. This arrangement is shown for illustration and is not limiting. Other arrangements of the stimulus presentation members are possible, including a co-axial arrangement. Further, the number of stimulus presentation members may be varied. Stimulus presentation members may be constructed out of the exemplar material, or they may be constructed of any other material on which the exemplar material may be displayed. Further, presentation of the stimulus exemplars is not limited to wheels; stimulus exemplars may be independently mounted or may be presented on disks, belts or a variety of different circular or polygonal shaped supports having flat, convex or concave surfaces. In addition, the number of exemplars per stimulus presentation member may be varied. For example, in FIG. 4, the stimulus wheels are shown with six exemplars per wheel for illustration only; exemplars may be added or removed. Moreover,

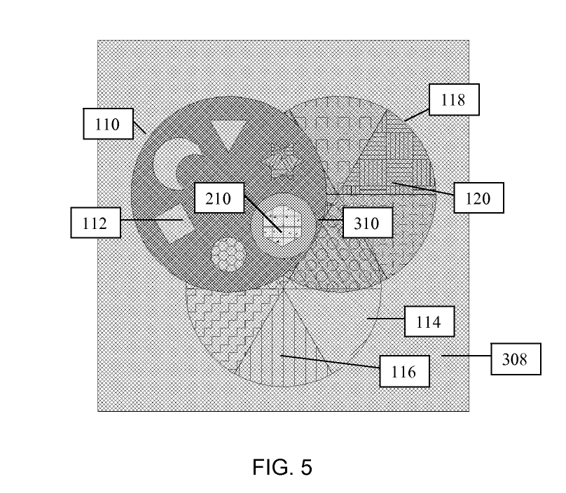

In a further aspect, the device 100 may include a stimulus focusing member over the stimulus presentation members for focusing the test subject's attention and facilitating access to the compound tactile stimulus 210. FIG. 5 illustrates one arrangement of the stimulus presentation members 110, 114, 118 behind a stimulus focusing member 306 comprised of a front panel 308 with an open window area 310. Generally, the panel will be opaque, but is shown in FIG. 5 as partially transparent for illustration purposes only. The panel 308 includes a window 310 restricted access by the test subject to only the portion of the wheels 210 behind the window 310 presenting the compound tactile stimulus 210 and providing access thereto. The window 310 may be centrally located. However, the location of the window and arrangement of the wheels in relation to the panel may be varied so long as the test subject may access the compound tactile stimulus 210 through the window 310 in the panel 308.

Automation means may be included to automate the movement and alignment of the stimulus presentation members in order to present a pre-selected compound tactile stimulus. The automation means may include means for determining the position of the stimulus presentation member, means for moving the stimulus presentation members, means for controlling the position and movement of the stimulus presentation members, signaling means for coordinating the activities of the automation means, and means for determining whether the compound tactile stimulus has been investigated by the animal.

Means for determining the position of stimulus presentation members may involve absolute position sensing, as well as indexed or limit position sensing. In absolute position sensing, each position is directly encoded, for example, digitally encoded by a rotational switch or analog encoded via a variable resistor. In indexed or limit position sensing, one (in the case of a rotational position) or two (in the case of linear positioning) positions are detected, and intermediate positions are located by counting the number of motor revolutions, stepper motor pulses, or solenoid ratchet actuations that have occurred. By way of example, if a stimulus presentation member or wheel requires 360 stepper motor pulses to complete one revolution, one could sense when the wheel is at “position zero” and can keep track of how many pulsed have been sent to the stepper motor to know the position of the stimulus presentation member or wheel.

Means for determining the position of stimulus presentation members may include one or more positional switches operatively linked to one or more stimulus presentation members. In addition, or alternatively, optical beam breaks, variable resistance, or electromechanical or optoelectrical encoders may be configured to monitor the position(s) of the stimulus presentation members. The positional switch(es) or other positional means may detect, for example, the position of a portion of the stimulus presentation member being presented at a pre-determined location, such as the window 310 in the front panel 308.

Means for moving or driving the stimulus presentation members may include one or more motors. Exemplary motors include but are not limited to a stepper motor, servo motor, solenoid drive, ratcheted solenoid drive, DC motor, pneumatic linear positioning, linear servo, linear stepper, and combinations thereof.

Means for controlling the position and movement of the stimulus presentation members may include the use of a computer, remote control device, or other suitable electronic device. The signaling means may employ interface electronics to coordinating the various activities of the automation means.

Means for determining whether the compound tactile stimulus has been investigated by the animal include an infra-red source operatively coupled to a detecting device and/or controlling device, such as a computer. The compound tactile stimulus device may be configured to allow the projection of an IR beam through one or more stimulus presentation members via channel(s) or through one or more clearance space(s) between one or more stimulus presentation members, such that when the animal investigates the presentation member(s) it breaks the IR beam(s). Alternative means for determining whether the compound tactile stimulus has been investigated by the animal include but are not limited to: switches, or pressure or force sensors actuated by the animal as it investigates the presentation members; vibrational (e.g. piezoelectric) or sound sensors detecting movement of the animal's appendages against the members; capacitance or resistance sensors that detect minute changes in the electrical properties of the presentation members when contacted by the animal.

As shown in FIG. 3, stimulus presentation members 110, 114, and 118 may be driven by a motor 122. Each stimulus presentation members may be driven by a separate motor 122. Alternatively, a plurality of stimulus presentation members may be driven by the same motor 122. The motor 122 may be controlled by a controlling device 126, such as a computer or other electronic devices, including but not limited to remote-control devices and the like. The controlling device 126 may send and receive signals from the device 100 through interface electronics 124. The interface electronics 124 may include wireless alternatives. The device 100 may include a base 130 and a membering structure 132 on which the components may be secured and brought into a preferred alignment.

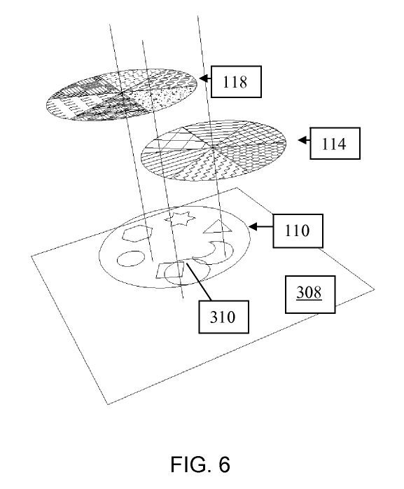

FIG. 6 illustrates a three dimensional exploded diagram of the overlapping of the stimulus wheels 110, 114, and 118 relative to the front panel 308. The stimulus wheels are drawn distanced from the neighboring wheels and the front panel for illustration only. The first 110, second 114, and third 118 stimulus wheels and the panel 308 may be mounted with the minimum possible clearance between each. The clearance between the stimulus wheels and the panel may vary. The stimulus wheels are shown as round; however, they may be oval, square, triangle, octagon, hexagon, pentagon, or any other smooth or multi-sided structure that can be rotated such that portions of each wheel overlap.

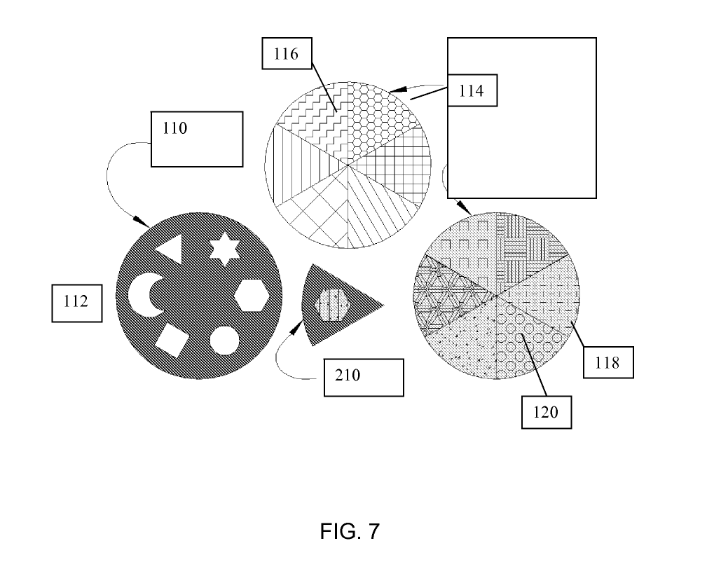

FIG. 7 is a detailed illustration of the first stimulus wheel 110, the second stimulus wheel 114, and the third stimulus wheel 118. The stimulus wheels may be freely removable from the device 100. The stimulus wheels may be customized by altering the dimensional spacing and exemplars within each dimensional plane.

In this example, the third stimulus wheel 120 may include various patterns 120. The patterns may be made of textured materials such as sandpaper, rubber, plaster, glass, wood, fiberboard, or other materials. The second stimulus wheel 114 may display variously arranged mesh bars 116. The mesh may be made of wire, plastic, rubber, glass, wood, fibers, or other materials. The first stimulus wheel 110 may display various shaped cut outs 112. A compound tactile stimulus 210 may result when the textured 120 third stimulus wheel 118 is overlaid with the mesh 116 second wheel 114, which is, in turn, overlaid with the shape cut-out 112 first wheel 110. The exemplar materials may be varied, as may the number and shape of the wheels. Further, the tactile stimuli may be combined with other stimuli such as odor, or visual stimuli. The wheels may be capable of overlapping, and all but the last wheel may have physical access to the wheel behind it.

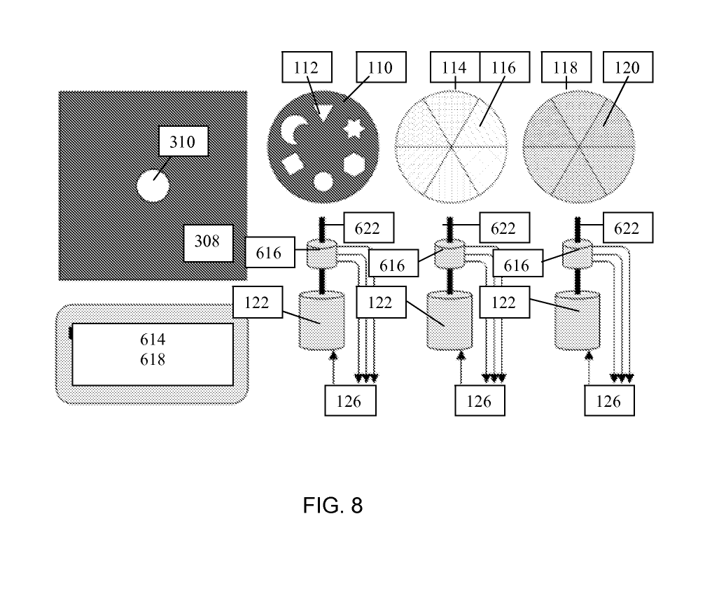

FIG. 8 is an exemplary view of components of an automated tactile stimulus presenting device. The components may include a front panel 308 which may define a window 310 through which a portion of the front-most stimulus wheel may be viewed or touched 210. Three stimulus wheels 110, 114, and 118 may each provide one of the dimensional planes of the compound tactile stimulus. A separate motor 122 that may move each wheel and a connected positional switch 616 that may detect which portion of the wheel is being presented through the front panel window 310. Basic interface electronics 614 and 618 may allow a controlling device 126 to control the stimulus wheels 110, 114, and 118 and monitor their position. The basic interface electronics may be combined or separated by function 614 and 618, such as a switch interface 614 and a motor interface 618.

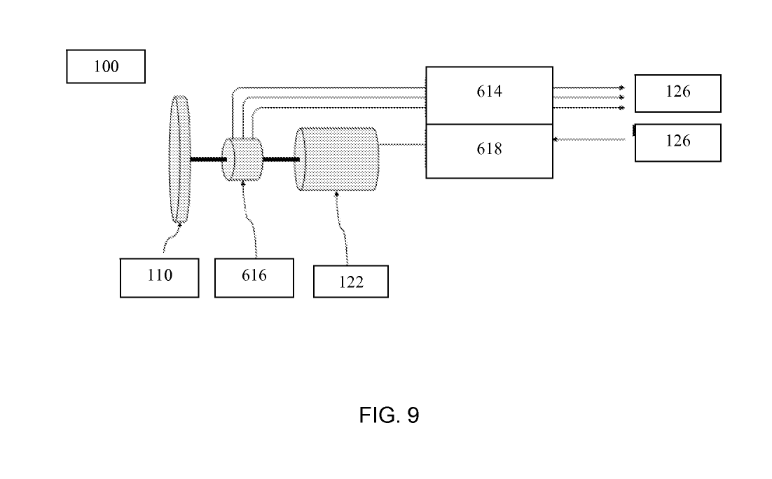

FIG. 9 is a second exemplary schematic view of the components for one stimulus wheel. This illustration is an example of one possible arrangement and is not limiting. The components may include: a stimulus wheel 110, a motor 122, a positional switch 616, basic interface electronics 614 and 618 and a controlling device 126. The stimulus wheel may be controlled by a stepper motor 122 that may directly drive both the stimulus wheel 110 and a positional switch 616. The positional switch 616 may monitor the position of stimulus presentation members or wheels 110. The position of the wheel may be relayed to a controlling device 126 by interface electronics 614. The controlling device 126 may adjust the position of the wheel by sending a signal to the interface electronics 618. The interface electronics 618 may turn the motors 122 which drive the wheels on and off. The motor 122 may be a stepper or servo motor, a solenoid drive, a ratcheted solenoid drive, a DC motor, or any other device capable of driving the wheel. Particularly, any device capable of driving the wheel 110 in a measured, incremental, and repeatable manner. The motor 122 may drive the stimulus wheel 110 by gears, chains, belts or a combination of these and other driving mechanisms. Similarly, the positional switch 616 may be driven by gears, chain, or belt or a combination of these and other driving mechanisms. The positional switch 616 may be oriented separately from the motor 122, incorporated directly into the motor 122, or eliminated from the device. Alternatively, the position of the stimulus wheel 110 may be calculated by the controlling device 126.

The stimulus wheels 110, 114, and 118 may be independently driven by separate motors 122 and may be arranged in an overlapping triangle. The stimulus wheels 110, 114, and 118 may alternatively be driven by the same motor 122 or any combination of motors 122. The stimulus wheels 110, 114, and 118 may be arranged on a single axis.

The stimuli exemplars may alternatively be displayed on a series of belts, or mounted independently and brought into place by solenoids, servos, or other actuators. The stimuli exemplars may be displayed on a combination of belts, wheels, and independent mounts.

Positional switches 616 may digitally encode the angle of rotation of a central shaft 622, and the angle of rotation of the stimulus wheel 110, 114, 118. The switch interface 614 may relay this information directly to the controlling device 126, with suitable signal conditioning. The motor interface 618 may provide simple signal conditioning, such as relay control, to turn the motor on and off given a suitable digital signal from the controlling device 126. The interface electronics may be simple.

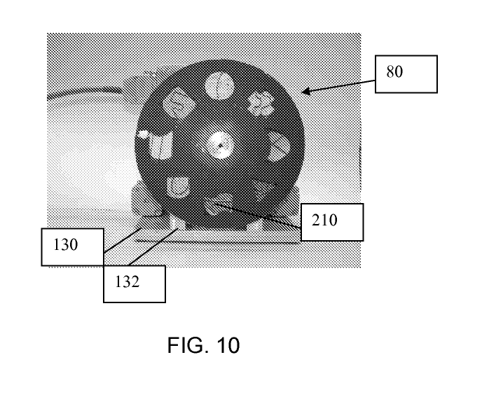

FIG. 10 illustrates another exemplary arrangement of stimulus wheels 80. In this configuration, the stimulus wheels are arranged co-axially.

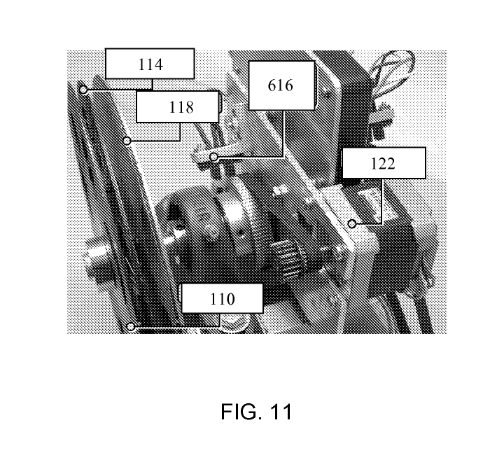

FIG. 11 illustrates a top view of the exemplary arrangement of FIG. 10. This figure is for illustrative purposes only and is not limiting. In this figure, stimulus wheels 110, 114, 118 are arranged co-axially. The arrangement includes an optional position counter 616. The position counter 616 may be, for example, an optical position counter. A motor 122, which may be, for example, a stepper motor, may drive the stimulus wheels 110, 114, 118. In FIG. 11, the clearance between the center 114 and back 118 wheels is greater than that between the front 110 and center 114 wheels. This facilitates the projection of an infra-red beam (not shown) between the center 114 and back 118 wheels, which provides a means for determining whether an animal has investigated a compound tactile stimulus.

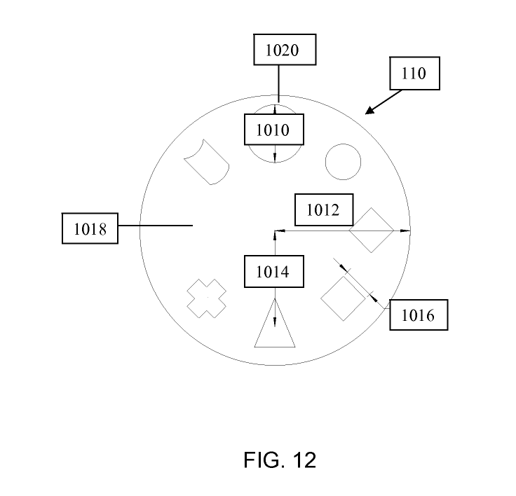

FIG. 12 is a detailed illustration of another possible arrangement of a first stimulus wheel 110. A first stimulus wheel 110 may display various cut out shapes. The wheel may contain a portion 1018 with no cut out which may represent a completely closed stimulus that may obstruct the wheel behind it. This portion 1018 may provide for a “control” condition. The wheel may contain a portion 1020 that is completely open. A completely open region 1020 may provide for a “control” condition. The detailed description of possible dimensions that follows is merely illustrative and not limiting.

Dimension 1010 may be 1.50 inches. Dimension 1012 may be 3.50 inches. Dimension 1014 may be 2.50 inches. Dimension 1016 may be 0.82 inches. These dimensions are illustrative only and may be tailored to the dimensions desired for the particular test subject and the experimental design.

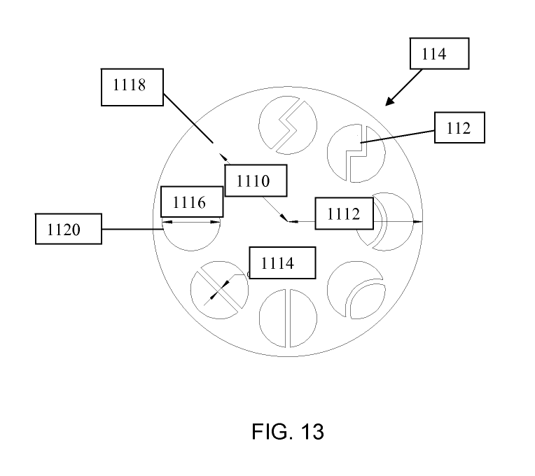

FIG. 13 is a detailed illustration of another possible arrangement of a second stimulus wheel 114. In a second stimulus wheel 114, the mesh may be replaced with a line stimulus 1120. The line stimulus 1120 may span a “window” in the stimulus wheel 114. The line stimulus 1120 may be a thin line of metal, plastic, fiber, glass, cardboard, fabric, composite, mixtures or combinations of the foregoing, and the like. The wheel may contain a portion 1118 with no cut out which may represent a completely closed stimulus that may obstruct the wheel behind it. This portion 1118 may provide for a “control” condition. The wheel may contain a portion 1120 that is completely open. A completely open region 1120 may provide for a “control” condition. The detailed description of possible dimensions that follows is merely illustrative and not limiting.

Dimension 1110 may be 2.50 inches. Dimension 1112 may be 3.50 inches. Dimension 1114 may be 0.125 inches. Dimension 1116 may be 1.50 inches. These dimensions are illustrative only and may be tailored to the dimensions required for the particular test subject and the experimental design.

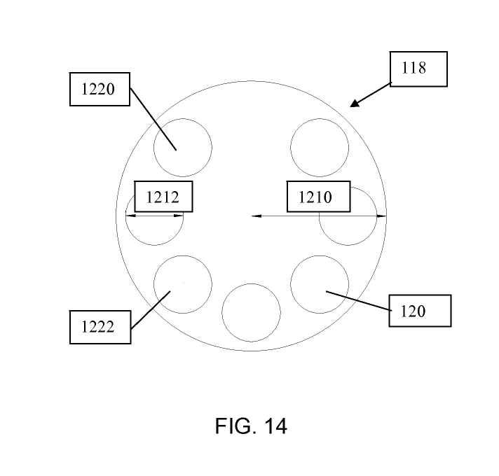

FIG. 14 is a detailed illustration of another possible arrangement of a third stimulus wheel 118. A third stimulus wheel 118 may present tactile stimuli in the form of multiple textured regions 120. There are several ways that the textured regions may be created. The following examples are merely illustrative and not limiting. The textured regions may be mounted in a series of “windows” 1222. Mounting may be achieved by, for example, sandwiching the textured region 120 between a first and second plate. Alternatively, the textured region may be etched directly into the wheel. The detailed description of possible dimensions that follows is merely illustrative and not limiting.

Dimension 1212 may be 1.50 inches. Dimension 1210 may be 3.50 inches. These dimensions are illustrative only and may be tailored to the dimensions desired for the particular test subject and the experimental design. A third may contain a portion 1220 that is completely open. A completely open region 1220 may provide for a “control” condition.

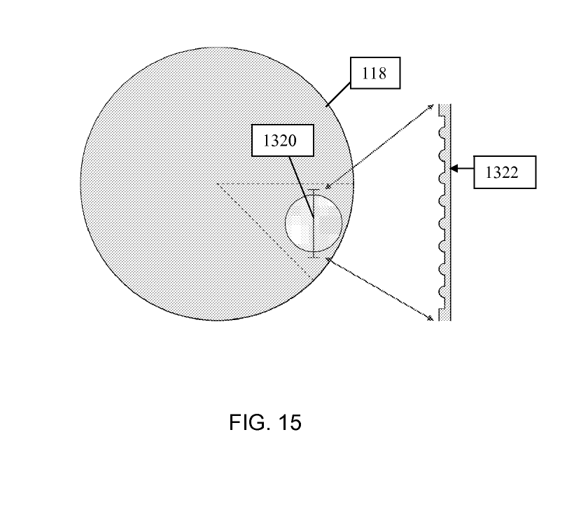

FIG. 15 is a detailed illustration of another possible arrangement of a third stimulus wheel 118. A third stimulus wheel 118 may present tactile stimuli in the form of multiple textured regions 1320. There are several ways that the textured regions may be created. The following example is merely illustrative and not limiting. The textured region may be etched directly into the wheel 1320. The etching may be achieved by, for example, a water jet cutter, a laser cutter, manual etching, chemical etching, a combination of these, or the like. An exploded view 1322 illustrates possible etching pattern. The etching may be limited to a “window” region, or it may cover an entire segment of a stimulus wheel 118.

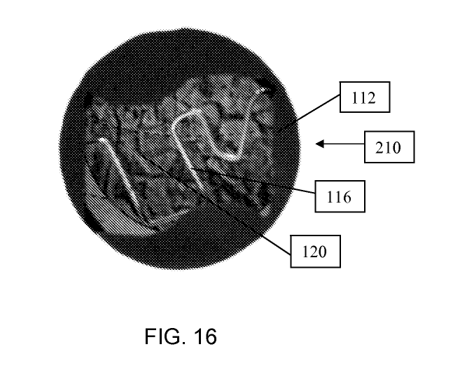

FIG. 16 illustrates an exploded view of a possible arrangement of overlapping tactile stimuli as they might be presented to a test subject. In this example, the textured material 120 may lie under a stimulus wire 116. A shaped cut out 112 may overlap tactile stimuli 120 and 116. The overlapping stimuli 112, 116 and 120 may create compound tactile stimuli. The arrangement of the wheels and the manner in which they are overlapped are for illustrative purposes only and are not limiting. The wheels may be arranged in other formats or sequence. Further, additional layers of stimuli may be added or removed.

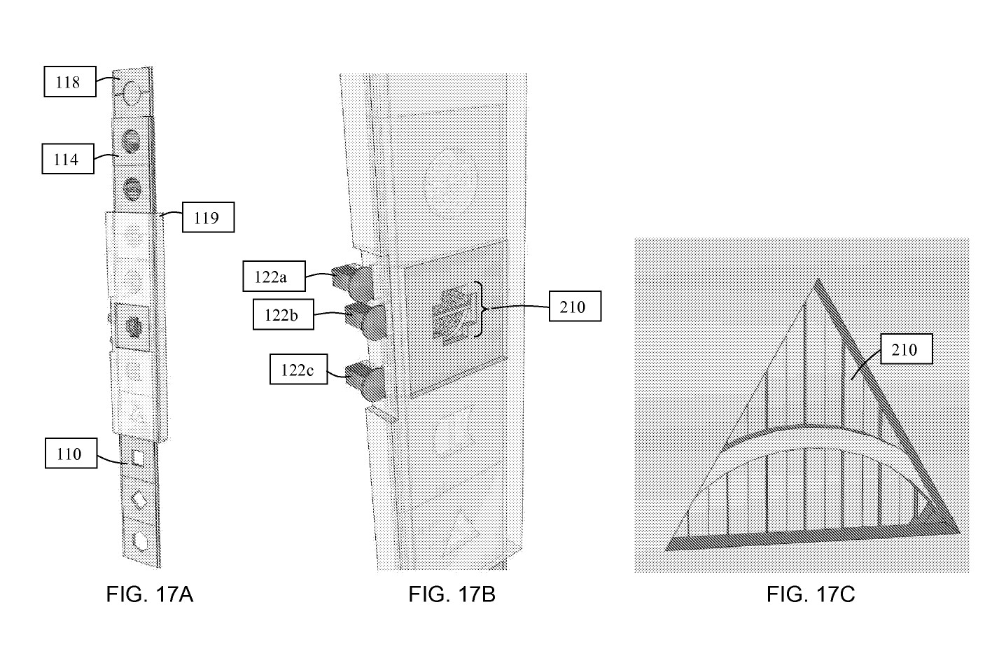

In another embodiment, the stimulus presentation members may be laterally extendable relative to one another. FIGS. 17A and 17B illustrate full view and close-up depictions, respectively, of laterally extendable stimulus presentation members as three plates 110, 114, and 118 arranged in an automated linear drive configuration. Each plate includes a different set of tactile stimulus exemplars 112, 116, and 120 analogous to those arranged in a circular fashion around the stimulus wheels depicted in FIGS. 5-8, 10, 12, and 13.

FIG. 17B shows a close-up of a compound tactile stimulus 210 formed by the overlap of three exemplars from three plates 110, 114, and 118. FIG. 17B further illustrates motors 122a, 122b, 122c for moving the plates 110, 114, and 118 independently of one another to produce the compound stimulus 210. An IR beam (not shown) is directed through a channel (not shown) in the frame 119 and plates 110, 114, and 118. The IR beam facilitates confirmation that the stimulus exemplars have been investigated by the animal. Indexing of the plates 110, 114, and 118 is provided by positional sensors in the frame 119 or motors 122a, 122b, 122c.

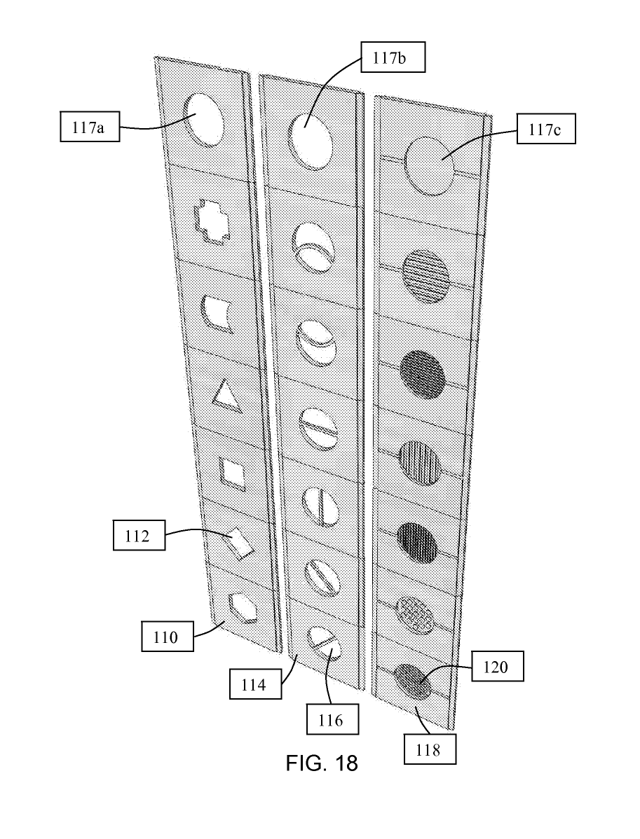

FIG. 18 illustrates the individual plates 110, 114, and 118 depicted in the linear drive configuration shown in FIGS. 17A and 17B. In FIG. 18, each plate includes a different set of 6 exemplars 112, 116, and 120 and a control 117a, 117b, and 117c. The plates 110, 114, and 118 are aligned in a frame 119. The center and back plates 114, 118, respectively, have an inlaid groove 125 that aligns to form a channel 127 for an IR beam 129 to detect investigation of the compound tactile stimulus 210 by the animal.

Automating compound tactile stimulus presentation can allow an experiment to proceed without experimenter intervention. For example, all of the test subject's food and water may be provided in the experimental set up through feeders in a maze. The test subjects may earn all of their food by running through the maze. This may allow the experimenter to collect data continuously throughout the test-subject's cycle without being present or physically intruding on the experimental design, or biasing the recovered data.

In a further aspect, the compound tactile stimulus presentation system may further include housing in which the compound tactile stimulus device is enclosed. In one embodiment, the system includes the compound tactile stimulus device integrated into an automated 24 hour a day living environment system providing automated compound tactile stimulus presentation. In another embodiment, the system provides an automated test environment in which pre-determined compound tactile stimuli, rewards, and/or punishments are presented under control of one or more controlling devices, and behavioral data is automatically generated and entered into one or more controlling devices, including but not limited to computers, data loggers, paper strip-charts or other electromechanical or mechanical records. Implementation of these systems enables animals to unwittingly participate in high-throughput behavioral studies during normally active time periods in which they are motivated to execute normal tasks and behaviors, such as foraging, whereby they work for their food and other resources, while generating behavioral data during their daily routine.

Animals with sensory modalities enhanced for discriminating tactile stimulus exemplars are especially suited for the above-described compound tactile stimulus presentation system, particularly animals with whiskers or fingers that may have relatively poor vision or hearing. Exemplary animals for discriminating tactile exemplars in a compound tactile stimulus include but are not limited to rodents, such as mice, and rats; guinea pigs; cats, dogs, primates, and also invertebrates with well developed tactile senses (such as cephalopods).

The systems and methods described herein are particularly suited for animals with relatively poor vision and hearing, such as mice. The use of mice and other small animals can further facilitate high-throughput testing involving between 80 to about 160 tests per day (for the average mouse), especially when providing small food rewards and short test cycles between about 10 to about 20 minutes, which can be regulated by programming access to one or more one-way doors under a pre-determined time sequence. The one-way doors may restrict access by being locked for a pre-determined time (for example, 15 minutes unlocked and then 15 minutes locked). Because mice have small stomachs, they can only eat small amounts of food at a time. Therefore, high-throughput testing may require limiting the food rewards to between about 0.015 and about 0.025 grams at a time, for example. This provides motivation for the mice to perform repeated tests, which serve to generate a significantly increased amount of test data relative to manual or automated systems conventionally used in the art.

In one embodiment, the housing includes a home cage for housing one or more animals. The home cage may be connected to one or more housing compartments or sub-compartments enclosing the compound tactile stimulus device. Housing compartments may include a maze, operant chamber, skinner box, or combinations thereof, any one of which may be divided into one or more sub-compartments. Housing compartments or sub-compartments may be further separated from another by one or more one-way doors, guillotine doors, trapdoors, or other doors or obstacles that can be opened and closed and/or locked to limit the direction of travel or the choices available to the animal.

In another embodiment, a compound tactile stimulus presentation system may further include means for delivering a reward or punishment, or neither, depending on the pre-determined compound tactile stimulus, and the animal's response thereto. The means for delivering rewards or punishments may include the use of controlling devices programmed to deliver the rewards or punishments, such as computers, remote control devices, or mechanical or electromechanical or electrical means directly operated by the experimenter. By way of example, the housing may include one or more feeders programmed under control of a controlling device, such as a computer, to deliver a pre-determined amount of a reward, such as food, water, drugs or other rewards under computer control.

Exemplary rewards include but are not limited to food, water, palatable liquid solution(s), and palatable solid pellet(s); rewarding drug(s), such as cocaine, alcohol, and nicotine; access to sexual partners or conspecifics; access to desired resources, such as nesting material(s), shelter, changes in ambient temperature, changes in ambient light; intercranial electrical or chemical stimulation of the brain; access to secondarily conditioned reinforcers (i.e., stimuli under a Pavlovian association with reward); shelter from aversive stimuli, such as uncomfortable temperatures, bright lights, loud noise, puffs of air, electric shock, or noxious odors; combinations thereof; and the like.

Exemplary punishments include but are limited to uncomfortable temperatures, bright lights, loud noise, puffs of air, electric shock, isolation from conspecifics, unpalatable liquids or solids, noxious odors (such as ammonia), noxious drugs, combinations thereof, and the like.

A system according to the present invention may include a plurality of compound tactile stimulus devices. Each device may be configured to independently present a pre-determined compound tactile stimulus to the animal indicating one or more locations that may or may not contain an accessible reward. Alternatively, the pre-determined compound tactile stimulus may indicate one or more locations that may or may not contain an accessible punishment. Further, the system may be configured so that multiple compound tactile stimuli are presented concurrently, sequentially, or in combination thereof.

Exemplary suppliers of animal housing materials and systems, including automated systems and devices, including but not limited to automated and non-automated housing supplies, feeders, response sensors, levers, one-way doors, and computer software for processes described herein include Coulbourn Instruments (Allentown, Pa.), TSE Systems, Inc. (Midland, Mich.), Lafayette Instruments (Lafayette, Ind.), Med Associates (St. Albans, Vt.), NewBehavior AG (Zurich, Switzerland), Lab Products, Inc. (Seaford, Del.), Alternative Designs Manufacturing and Supply, Inc. (Siloam Springs, Ark.), and Bio-Sery (Frenchtown, N.J.).

As noted above, the compound tactile stimulus presentation system of the present invention can facilitate high-throughput behavioral studies in the fields of animal behavior (ethology), experimental psychology, neurobiology, drug development, pharmacology, genetic engineering, and psychiatric research.

In one embodiment, a method for conducting an animal test includes exposing the animal to a compound tactile stimulus presentation system according to the present invention so that at least one pre-determined compound tactile stimulus is presented to the animal, whereby the stimulus indicates one or more locations that may or may not contain an accessible reward, punishment, or neither. Conditions suitable for enabling the animal to receive the reward, punishment, or neither, depending on the animal's response to the compound stimulus are provided. These steps are repeated, altering the compound tactile stimulus as appropriate until a suitable number of tests (or cycles of the above steps) have been obtained in accordance with the underlying behavioral test or behavioral paradigm(s) which are being investigated in accordance the methodologies known to those of skill in the art. Alternatively, the conditions suitable for enabling receipt of the reward, punishment, or neither, may depend on the animal's response to multiple independent compound tactile stimuli presented concurrently, sequentially, or combination thereof, from a plurality of compound tactile stimulus devices set up in one cycle of steps, which may be repeated until a suitable number of cycles have been obtained as deemed by those of skill in the art.

In another embodiment, a method for testing an animal includes initiating a test by placing an animal in a home cage containing a first one-way door leading to a first compartment containing a compound tactile stimulus presentation device according to the present invention. The device may be positioned between a second one-way door leading into a second compartment and a third one-way door leading into a third compartment, whereby one or both of the first and second compartments contains a reward, punishment, or neither. The animal enters through the first one-way door into the first compartment where the animal is exposed to the device presenting a pre-determined compound tactile stimulus indicating the location(s) of accessible reward(s) or punishments(s) in the second compartment, the third compartment, or both. The animal investigates the stimulus and chooses whether to enter the second or third compartment through its corresponding second or third one-way door, respectively. Upon entry into either of the second or third compartments and ingesting food if it is accessible, the animal is allowed to exit the compartment through either a fourth or fifth one-way door leading into a home cage. The second compartment leads to the home cage through the fourth one-way door and the third compartment leads to the home cage through the fifth one-way door. Once the animal enters the home cage, the animal can repeat the above steps multiple times, whereupon in each case the compound tactile stimulus may remain the same or it may be changed. Similarly the compartment containing accessible food may remain the same or it may be changed, depending on the compound tactile stimulus. Preferred animal test methods include a step for determining whether a compound stimulus has been investigated by an animal. This may involve the use of infra-red beams as described above.

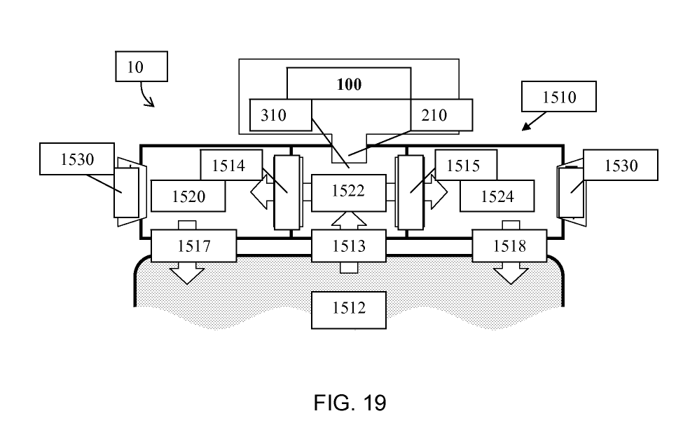

FIG. 19 is an exemplary diagram illustrating a compound tactile device system 10 in a maze paradigm experiment. The device 100 may be enclosed in a maze 1510 such that a test subject can access the compound tactile stimulus 210 through the window 310 of the device's front panel 308. This exemplary maze paradigm experiment illustrates a single trial in a learning, cognitive, or neuropsychological paradigm employing a tactile stimulus presentation device 100. The experiment is illustrative and not limiting. In this experiment, the maze is configured as a long, interrupted corridor attached to the side of a home cage 1512, and joined therebetween by a first one-way door 1513. The corridor may be divided into three compartments, a left compartment 1520, a central compartment 1522, and a right compartment 1524. A second one-way door 1514 connects the central compartment to the left compartment 1520. A third one-way door 1515 connects the central compartment to the right compartment 1520. A test subject may enter the central compartment 1522 via the first one-way door 1513 from the home cage 1512. The test subject may investigate the tactile stimulus 210 presented by the device 100 in the central compartment 1522. The test subject may then choose either the left compartment 1520 by entering through the second one-way door 1514 or the right compartment 1524 by entering through the third one-way door 1515. The left compartment 1520 and the right compartment 1524 may each contain a feeder 1530. One or both of the feeders 1530 may deliver a small amount of food under computer control. Thus one tactile stimulus 210 might indicate that food will be delivered to the feeder 1530 in the right compartment 1524, while another tactile stimulus 210 might indicate that the food will be delivered to the feeder 1530 in the left compartment 1520. Alternatively, the tactile stimulus 210 may indicate that more food will be delivered by one feeder 1530 as opposed to the other, or that there is a greater probability of one feeder 1530 delivering food than the other feeder 1530. The test subject may then return to the home cage 1512 through a fourth 1517 or fifth 1518 one-way door. Once back in the home cage 1512, the animal can repeat the above steps, in which case the same compound tactile stimulus 210 may be retained or modified in any subsequent trial.

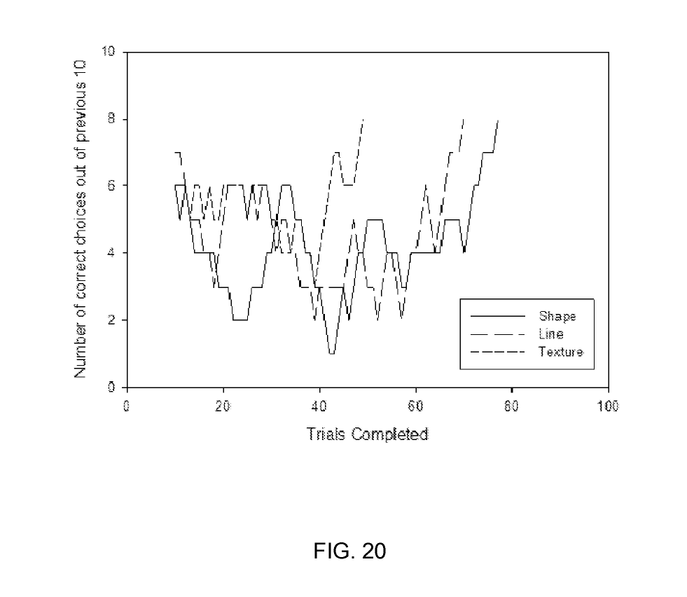

FIG. 20 illustrates the results of a simple mouse tactile discrimination test (3 mice) in which tactile exemplars in each of three dimensions (shape, line, texture) were tested in isolation using the automated device in FIG. 11 in the maze system described in FIG. 19. In each test one exemplar instructed the mouse to go right and the other exemplar instructed the mouse to go left. The graph plots number of correct choices out of previous 10 trials (y-axis) as a function of trials completed (x-axis). The data shows that mice tend to initially choose at random, but then significantly improve at picking correctly after about 40 to 70 trials.

In any one of the above described compound tactile stimulus devices or systems, the compound tactile stimulus presented may: (1) indicate the location of a reward (or punishment) in one compartment and no reward (or no punishment) in another compartment; (2) indicate the location of a reward in one compartment and a punishment in another compartment; (3) indicate differing amounts of the reward (or punishment) in one compartment relative to another compartment; (4) provide a relative probability that a reward (or punishment) is more likely to be present in one compartment relative to another compartment; (5) indicate the relative degree of delay in obtaining a reward (or punishment) in a particular compartment, whereby the delay is concluded and the reward (or punishment) is administered or made available after a set period of time, or only upon completion of a further task by the animal; (6) indicate the relative degree of delay in obtaining a reward (or punishment) in a particular compartment relative to another compartment. In the case of (5) or (6), the delay may be concluded and the reward (or punishment) may be administered or made available after a set period of time, or only upon completion of a further task by the animal.

By way of example, a tactile stimulus exemplar in the form of a horizontal line (or 0 degree angle) may indicate the location of a reward in a first compartment only (and no reward in a second compartment, for example), whereas a vertical line (or 90 degree angle) may indicate the location of a reward in the second compartment only (and no reward in the first compartment, for example). Further, with respect to the above example, a tactile stimulus exemplar in the form of a line at a 45 degree angle may indicate an equal probability of a reward in either of the two compartments (alone), or it may indicate no increased delay in receiving a reward in one compartment relative to another. In contrast, a tactile stimulus exemplar in the form of a line at a 60 degree angle may indicate an increased probability of a reward in the second compartment only (relative to a another compartment) or it may indicate a reduced delay in receiving a reward in the second compartment relative to the first compartment.

The systems of the present invention may be used in animal behavioral tests to evaluate any one of a variety of learning, cognitive, or neuropsychological paradigms known to those of skill in the art. Presentation of compound tactile stimuli by the inventive device of the present invention is particularly suited for behavioral testing, including but not limited to: investigation of animals with defects affecting cognitive function; screening animals for behavioral mutants; screening genetically engineered animals for behavioral phenotypes; comparing behavioral results obtained between animals exposed to a test drug and control animals not exposed to the drug; reverse-translational paradigms homologous to a cognitive neuropsychological or memory task used in human research or diagnosis; reverse-translational paradigms(s) homologous to a biomarker(s) or endophenotype(s) of human disorders or disease processes; diagnosis of neuropsychological behaviors or disorders in an animal; investigation of abnormal repetitive behaviors; testing of affective-shifting, set-shifting, and response-shifting; measuring perseveration or learned irrelevance; testing of delay discounting or other measures of impulsivity; testing of cognitive bias or other measures of catastrophic thinking or depressed affect; testing of rewarded alteration or other measures of hippocampus dependent and hippocampus independent working memory; testing of delayed match-to-sample, delayed alternation, or other measures of working memory.

The above described steps or tasks may are formulated to model complex combinations of paradigms presenting a battery of tasks to provide an assay of cognitive, neuropsychological, or memory functions. For example, the steps may formulated as combinations of paradigms to present a battery of tasks for diagnosing between hippocampus dependent working memory and hippocampus independent working memory, or for diagnosing between perseverative and learned irrelevance deficits in set shifting.

### EXAMPLES

### Example 1

Operant and maze tasks in mice are limited by the small number of trials (˜15) possible in a session before they lose motivation. It was hypothesized that by manipulating reward size and session length, motivation, and hence performance, would be maintained in an automated T-maze. It was predicted that larger rewards and shorter sessions would improve acquisition; and that smaller rewards and shorter sessions would maintain higher and less variable performance. 18 C57BL/6 mice (9 males and 9 females) acquired (criterion 8/10 correct) and performed a spatial discrimination, with one of 3 reward sizes (0.02, 0.04, or 0.08 g) and one of 3 session schedules (15, 30, or 45 minute sessions with the same inter-session intervals). Each mouse had a total of 360 minutes of access to the maze per night, for two nights, and completed 190 trials on average. Analysis used split-plot GLM with contrasts testing for linear effects. Acquisition of the discrimination was unaffected by reward size or session length/interval. As predicted, after-criterion average performance improved as reward size decreased (p<0.001). As predicted, after-criterion variability in performance increased as reward size increased (p=0.001). Session length/interval did not affect any outcome. It was concluded that an automated maze, with suitable reward sizes, can provide sustained performance with low variability, at 5-10 times the throughput of traditional methods.

### Example 2

Memory deficits associated with hippocampal dysfunction are a key feature of a number of neurodegenerative and psychiatric disorders. Discrete-trial rewarded alternation task in a T-maze is extremely sensitive to hippocampal dysfunction. Each trial of the rewarded alternation comprises a sample run and a choice run. On the sample run, the mouse can only enter one of the goal arms (chosen at random) to get a reward; the other arm being closed. On the choice run, the mouse is allowed a free choice of either arm, but is rewarded for choosing the arm which it did not visit in the sample run. Previous studies have shown that sham mice have spontaneously high levels of alternation, whereas hippocampal lesioned mice exhibit chance levels of performance (50%). However, the task is hand-run, requires extensive habituation, and thus is very labour-intensive.

In this example, bilateral hippocampal-lesioned mice were used to validate the discrete-trial rewarded alternation paradigm using an automated T-maze system. Briefly, a fully automated T-maze was attached to a plastic home cage. To reduce the anxiety a companion mouse was paired with the test subject. The cage was divided by a partition which allows physical, auditory, and olfactory contact between the “test mouse” and the “companion mouse”. The companion mouse, which was not tested, was provided with ad libitum food and water. The test mouse was provided with free access to water only and earns all of its food within the maze. Each component of the automated T-maze was individually and independently controlled by a computer.

Two versions of the task (i.e. the automated T-maze and the hand-run standard T-maze) were run in parallel according to a cross-over design. The results showed a significant effect of lesion (p<0.05) in both versions of the maze, confirming that the automated version of discrete-trial rewarded alternation is dependent on an intact hippocampus. Accordingly, this system provides a high throughput assay of hippocampal function suitable for drug discovery

While various embodiments of the invention have been described, it will be apparent to those of ordinary skill in the art that many more embodiments and implementations are possible that are within the scope of the invention.

1-86. (canceled)

87. A system for presenting compound tactile stimuli to a test subject, the system comprising a compound stimulus device comprising:
a plurality of overlapping stimulus presentation members, each stimulus presentation member comprising one or more stimulus exemplars related by a specified dimensional property, including at least one exemplar comprising one or more open spaces;
means for moving the stimulus presentation members relative to one another, aligning the exemplars from one presentation member with exemplars in other presentation member(s) to form a pre-determined compound tactile stimulus;
wherein the compound tactile stimulus is presented so that the test subject can investigate the stimulus exemplars underlying the compound stimulus by extending one or sensory members through the one or more open spaces.

88. The system of claim 87, wherein the compound stimulus comprises front, center, and back tactile stimulus exemplars presented from front, center, and back stimulus presentation members, wherein each of the front and center tactile stimulus exemplars comprises one or more open spaces.

89. The system of claim 87, wherein the compound tactile stimulus device comprises automation means for automating the movement and alignment of the stimulus presentation members to present a pre-selected compound stimulus.

90. The system of claim 89, wherein the automation means comprises:
positional means for determining the position of the stimulus presentation members;
driving means for driving the stimulus presentation members;
controlling means for controlling the position and movement of the stimulus presentation members; and
signaling means for coordinating execution of the positional, driving, and controlling means.

91. The system of claim 90, wherein the automation means further comprises investigation means for determining whether the compound tactile stimulus has been investigated by an animal.

92. The system of claim 90, wherein the means for driving the stimulus presentation members comprise a gear, chain, belt, worm, drive, ratchet, pneumatic, linear stepper, linear actuator, or combination thereof.

93. The system of claim 89, wherein the compound tactile stimulus device comprises:
one or more motors, each motor driving one or more of the stimulus presentation members;
a controlling device operatively coupled to the one or more motors; and
interface electronics operatively coupled to the controlling device, the interface electronics coordinating signals between the controlling device and the one or more motors to control movement of the stimulus presentation members.

94. The system of claim 87, further comprising housing in which the compound tactile stimulus device is enclosed.

95. The system of claim 94, further comprising a home cage for housing one or more animals.

96. A method for conducting an animal test comprising the steps of:
(a) exposing the animal to the system of claim 94, wherein at least one compound tactile stimulus device presents a pre-determined compound tactile stimulus to the animal indicating:
(i) one or more locations that may or may not contain an accessible reward, and/or
(ii) one or more locations that may or may not contain an accessible punishment;
(b) providing conditions suitable for enabling the animal to receive the reward, punishment, or neither, depending on the animal's response to the compound stimulus, and
(c) repeating steps (a) and (b) until a suitable number of tests have been obtained.

97. The method of claim 96, further comprising the step of determining whether the animal has investigated the compound tactile stimulus.

98. The method of claim 96, wherein the compound stimulus comprises front, center, and back tactile stimulus exemplars presented from front, center, and back stimulus presentation members, wherein each of the front and center tactile stimulus exemplars comprises one or more open spaces.

99. The method of claim 96, wherein the animal is a rodent.

100. The method of claim 99, wherein the animal is a mouse.

101. The method of claim 100, wherein the test is repeated between about 80 to about 160 times a day.

102. The method of claim 96, wherein the animal has a defect affecting its cognitive function.

103. The method of claim 96, wherein the steps are formulated to screen genetically modified animals for behavioral phenotypes.

104. The method of claim 96, wherein the steps are formulated for a member of the group consisting of testing of affective-shifting, testing of setshifting, testing of response-shifting, measuring perseveration, measuring learned irrelevance, and combinations thereof.

105. The method of claim 96, wherein the steps are formulated for testing of delay discounting or other measures of impulsivity.

106. The method of claim 96, wherein the steps are formulated for testing of cognitive bias or other measures of depressed affect.
